(19) United States
(12) Patent Application Publication
(10) Pub. No.: US 2022/0273185 A1
(43) Pub. Date: Sep. 1, 2022

(54) ABDOMEN SCANNING METHOD AND SYSTEM, AND STORAGE MEDIUM

(71) Applicant: Siemens Healthcare GmbH, Erlangen (DE)

(72) Inventors: Liu En Yang, Shenzhen (CN); Yuan Yuan Kang, Shenzhen (CN); Fang Dong, Shenzhen (CN); Le Zhang, Shenzhen (CN)

(73) Assignee: Siemens Healthcare GmbH, Erlangen (DE)

(21) Appl. No.: 17/680,130

(22) Filed: Feb. 24, 2022

(30) Foreign Application Priority Data
Feb. 26, 2021 (CN) 202110217225.2

(51) Int. Cl. A61B 5/055; G06T 7/66; G06T 7/70; G06T 7/00; A61B 5/00

(52) U.S. Cl. CPC A61B 5/055; G06T 7/66; G06T 7/70; G06T 7/0012; A61B 5/4244; G06T 2207/20081; G06T 2207/30056; G06T 2207/10088; A61B 2576/02

(57) ABSTRACT

Abdomen scanning techniques are disclosed. The techniques include acquiring a current abdomen positioning image; inputting the current abdomen positioning image into a pre-trained liver outline acquisition model, and obtaining current liver outline information outputted by the liver outline acquisition model; the liver outline acquisition model being obtained by training, using a first quantity of historical abdomen positioning images as an input set, and using a first quantity of historical liver outline information respectively corresponding to the first quantity of historical abdomen positioning images as an output set; based on the current liver outline information, calculating an abdomen scanning range and a scanning center position, and applying the scanning range and scanning center position in a current scanning protocol; and using the scanning protocol to perform a magnetic resonance scan of an abdomen.

### CROSS-REFERENCE TO RELATED APPLICATIONS

The present application claims the benefit of the filing date of China patent application no. CN 202110217225.2, filed on Feb. 26, 2021, the contents of which are incorporated herein by reference in their entirety.

### TECHNICAL FIELD

The disclosure relates to the field of medical equipment and, in particular, to an abdomen scanning method, system, and a computer readable storage medium.

### BACKGROUND

Magnetic resonance imaging (MRI) is a technology in which the phenomenon of magnetic resonance is used to perform imaging. In MRI systems, in an existing workflow for automated scanning of the abdomen, a situation often arises in which too large a scanning range is automatically selected in a direction from the head to the feet of the human body and in the anterior-posterior direction thereof. Too large a scanning range will inevitably increase the scanning time, making the patient less comfortable and less cooperative, and thus might result in reduced quality in the image obtained by scanning. Moreover, since the increased scanning range exceeds the region of interest, it cannot provide any more diagnostic information.

### SUMMARY

In view of the above, embodiments of the present disclosure propose an abdomen scanning method in one aspect, and in other aspects an abdomen scanning system and a computer readable storage medium for increasing the accuracy of automatic identification of an abdomen scanning range.

The abdomen scanning method proposed in embodiments of the present disclosure comprises: acquiring a current abdomen positioning image; inputting the current abdomen positioning image into a pre-trained liver outline acquisition model, and obtaining current liver outline information outputted by the liver outline acquisition model; the liver outline acquisition model being obtained by training, using a first quantity of historical abdomen positioning images as an input set, and using a first quantity of historical liver outline information respectively corresponding to the first quantity of historical abdomen positioning images as an output set; based on the current liver outline information, calculating an abdomen scanning range and a scanning center position, and applying the scanning range and scanning center position in a current scanning protocol; and using the scanning protocol to perform a magnetic resonance scan of an abdomen.

In one embodiment, before using the scanning protocol to perform a magnetic resonance scan of an abdomen, the method further comprises: calculating a liver center-of-mass position or a liver outline incircle center position based on the current liver outline information, calculating a navigation bar position required for respiratory navigation according to the liver center-of-mass position or the liver outline incircle center position, and applying the navigation bar position in the current scanning protocol.

In one embodiment, the abdomen positioning image comprises a sagittal image, a transverse image, and a coronal image; and the liver outline information comprises: a dimension in the head-feet direction, a dimension in the anterior-posterior direction, and a dimension in the left-right direction, taking the patient as the positioning center.

The abdomen scanning system proposed in embodiments of the present disclosure comprises: an image acquisition module configured to acquire a current abdomen positioning image; a liver outline information acquisition module configured to input the current abdomen positioning image into a pre-trained liver outline acquisition model, and obtain current liver outline information outputted by the liver outline acquisition model; the liver outline acquisition model being obtained by training, using a first quantity of historical abdomen positioning images as an input set, and using a first quantity of historical liver outline information respectively corresponding to the first quantity of historical abdomen positioning images as an output set; and a scanning range determining module configured to calculate an abdomen scanning range and a scanning center position based on the current liver outline information, and apply the scanning range and scanning center position in a current scanning protocol so that an MRI device can use the scanning protocol to perform a magnetic resonance scan of an abdomen.

In one embodiment, the system further comprises: a navigation bar position determining module configured to calculate a liver center-of-mass position or a liver outline incircle center position based on the current liver outline information, calculate a navigation bar position required for respiratory navigation according to the liver center-of-mass position or the liver outline incircle center position, and apply the navigation bar position in the current scanning protocol.

In one embodiment, the abdomen positioning image comprises a sagittal image, a transverse image, and a coronal image; and the liver outline information comprises: a dimension in the head-feet direction, a dimension in the anterior-posterior direction, and a dimension in the left-right direction, taking the patient as the positioning center.

Another abdomen scanning system proposed in embodiments of the present disclosure comprises: at least one memory and at least one processor, wherein: the at least one memory is configured to store a computer program; the at least one processor is configured to execute the computer program stored in the at least one memory, and to perform the abdomen scanning method as described in any one of the embodiments above.

A computer readable storage medium proposed in embodiments of the present disclosure has a computer program stored thereon; the computer program is executable by a processor to functionally realize the abdomen scanning method as described in any one of the embodiments above.

It can be seen from the solution above that in embodiments of the present disclosure, a liver outline acquisition model is obtained by training in advance based on an artificial intelligence network using a first quantity of historical abdomen positioning images as an input set, and using a first quantity of historical liver outline information respectively corresponding to the first quantity of historical abdomen positioning images as an output set, and a liver region outline information is acquired based on the liver outline acquisition model; and a scanning range is determined based on the acquired liver region outline information. As the liver outline information acquired by such a method is relatively accurate, it can increase the accuracy of automatic identification of the abdomen scanning range.

In addition, by determining a liver center-of-mass position or a liver outline incircle center position based on the acquired liver region outline information, and determining a navigation bar position based on the liver center-of-mass position or liver outline incircle center position, it is possible to ensure that the navigation bar position is within the outline of the liver region, thus increasing the accuracy of navigation bar positioning.

### BRIEF DESCRIPTION OF THE DRAWINGS/FIGURES

Preferred embodiments of the present disclosure are described in detail below with reference to the accompanying figures, to give those skilled in the art a clearer understanding of the abovementioned and other features and advantages of the present disclosure. In the figures:

FIG. 1 illustrates a demonstrative flow chart of an abdomen scanning method in an embodiment of the present disclosure.

FIG. 2A illustrates a schematic figure of a transverse image in an embodiment of the present disclosure.

FIG. 2B illustrates liver outline information that is output on the basis of the transverse image shown in FIG. 2A.

FIG. 2C illustrates liver outline information of FIG. 2B showing the incircle.

FIG. 3 illustrates a demonstrative structural diagram of an abdomen scanning system in an embodiment of the present disclosure.

FIG. 4 illustrates a demonstrative structural diagram of another abdomen scanning system in an embodiment of the present disclosure.

### KEY TO THE FIGURES

| Label | Meaning |
|---|---|
| 101-105 | steps |
| 1, 2 | boxes |
| 301 | image acquisition module |
| 302 | liver outline information acquisition module |
| 303 | scanning range determining module |
| 41 | memory |
| 42 | processor |
| 43 | bus |

### DETAILED DESCRIPTION

In embodiments of the present disclosure, taking into account the fact that an MR-scanned abdomen positioning image contains liver information, abdomen positioning is generally performed based on liver position. At present, the method of manual labelling is mainly used to determine the outline of the liver, and the scanning range (Field of View, “FOV”) of the region of interest of the abdomen is then calculated. If the positioning image is of high quality, an existing Snake image segmentation algorithm (Snake algorithm) can obtain a relatively accurate scanning range. If the positioning image is of relatively poor quality, the calculated range will be too large in the direction from the head to the feet of the human body and in the anterior-posterior direction thereof. In addition, in ordinary abdomen scanning, the operator must arrange the navigation bar according to the position of the liver, so as to acquire stable respiration information for triggering scanning. The method currently used is to fix the position of the navigation bar to the left of the center of the FOV. Although this method can cover the position of the liver in most situations, none of these positions are optimal and might deviate from the liver, so the respiration signal still has room for improvement. Furthermore, with this method, when the scanning layer direction is coronal situations will arise in which the navigation bar is not on the liver, leading to errors in the respiration signal, so that obvious respiration artifacts appear in the image.

To increase the accuracy of automatic identification of the abdomen scanning range, the embodiments described herein consider obtaining a liver outline acquisition model by training based on an artificial intelligence network to acquire outline information of a liver region based on the model. As the liver outline information acquired by such a method is relatively accurate, it can increase the accuracy of automatic identification of the abdomen scanning range.

In order to clarify the object, technical solution, and advantages of the present disclosure, the present disclosure is explained in further detail below by way of embodiments.

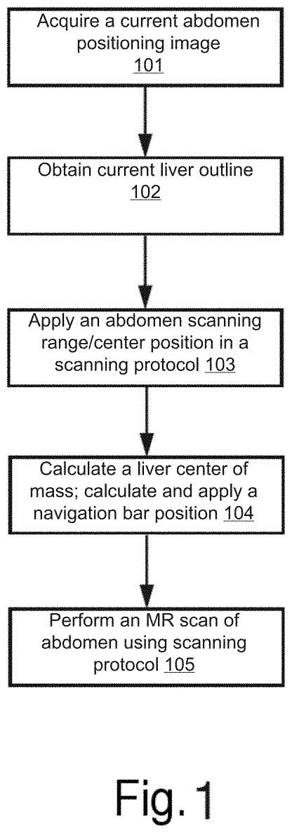

FIG. 1 illustrates a demonstrative flow chart of an abdomen scanning method in an embodiment of the present disclosure. As FIG. 1 shows, the method may comprise the following steps:

Step 101: acquiring a current abdomen positioning image.

In this embodiment, the abdomen positioning image may be three-plane positioning, e.g. may comprise: a sagittal image, a transverse image, and a coronal image.

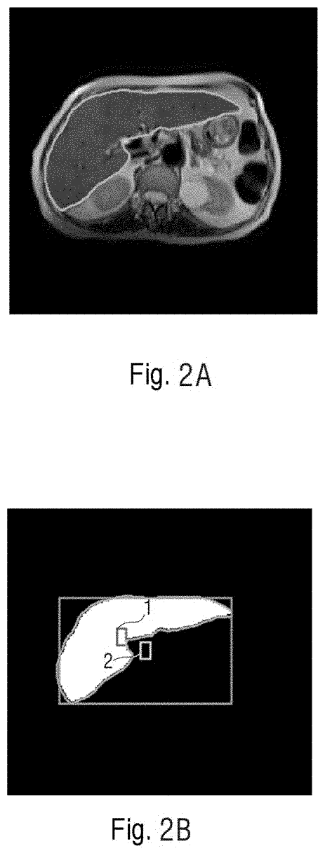

FIG. 2A illustrates a schematic figure of a transverse image in an embodiment of the present disclosure.

Step 102: inputting the current abdomen positioning image into a pre-trained liver outline acquisition model, and obtaining current liver outline information outputted by the liver outline acquisition model.

The liver outline acquisition model may be obtained by training, using a first quantity of historical abdomen positioning images as an input set, and using a first quantity of historical liver outline information respectively corresponding to the first quantity of historical abdomen positioning images as an output set. Here, the historical liver outline information may be liver outline information obtained based on manual labelling of abdomen positioning images.

The liver outline information may comprise: a dimension in the head-feet (HF) direction, a dimension in the anterior-posterior (AP) direction, and a dimension in the left-right (LR) direction, taking the patient as the positioning center. The dimension in the head-feet direction may be determined based on a transverse image, the dimension in the anterior-posterior direction may be determined based on a coronal image, and the dimension in the left-right direction may be determined based on a sagittal image.

FIG. 2B illustrates liver outline information outputted on the basis of the transverse image shown in FIG. 2A. FIG. 2C illustrates liver outline information of FIG. 2B showing the incircle in red with a center dot identifying the center position of the incircle. The incircle may be of any suitable size and have a center position that may be different than the center-of-mass position as represented in box 2. The incircle may be determined based upon any suitable geometric arrangement, e.g. a liver outline incircle center position wholly contained within the liver tissue with edges in contact with the outer edges of the liver tissue as shown in FIG. 2C.

Step 103: based on the current liver outline information, calculating an abdomen scanning range and a scanning center position, and applying the scanning range and scanning center position in a current scanning protocol.

Box number 1 in FIG. 2B is the scanning center position, and is also the position of the geometric center of the liver region.

Step 104: based on the current liver outline information, calculating a liver center-of-mass position or a liver outline incircle center position, calculating a navigation bar position required for respiratory navigation according to the liver center-of-mass position or the liver outline incircle center position, and applying the navigation bar position in the current scanning protocol.

In this step, the navigation bar position can be set at the center-of-mass position or at a position separated from the center-of-mass position by a set distance, or can be set at the liver outline incircle center position or at a position separated from the incircle center position by a set distance, etc.

Box number 2 in FIG. 2B is the liver center-of-mass position.

Step 105: using the scanning protocol to perform a magnetic resonance scan of an abdomen.

In this embodiment, there is no absolute order for performing step 103 and step 104. For example, it is also possible to perform step 104 first and then perform step 103, or step 103 and step 104 can be performed at the same time. Of course, in other embodiments, the navigation bar position may also be determined using an existing method; for example, the navigation bar position is still fixed at a position to the left of the center of the FOV, etc. In addition, the navigation bar position may also be determined by other methods, which are not defined here.

The abdomen scanning method in embodiments of the present disclosure has been described in detail above; the abdomen scanning system in embodiments of the present disclosure is described in detail below. The abdomen scanning system in embodiments of the present disclosure can be used to implement the abdomen scanning method in embodiments of the present disclosure. For particulars not disclosed in detail in system embodiments of the present disclosure, reference can be made to the corresponding description in method embodiments of the present disclosure; an item-by-item description is not repeated here.

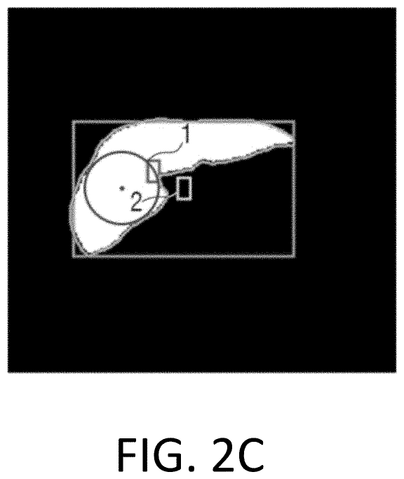

FIG. 3 illustrates a demonstrative structural diagram of an abdomen scanning system in an embodiment of the present disclosure. As shown in FIG. 3, the system may comprise the following, as shown by the solid-line parts in FIG. 3: an image acquisition module 301, a liver outline information acquisition module 302 and a scanning range determining module 303.

The image acquisition module 301 is configured to acquire a current abdomen positioning image.

The liver outline information acquisition module 302 is configured to input the current abdomen positioning image into a pre-trained liver outline acquisition model, and to obtain current liver outline information output by the liver outline acquisition model; the liver outline acquisition model is obtained by training, using a first quantity of historical abdomen positioning images as an input set, and using a first quantity of historical liver outline information respectively corresponding to the first quantity of historical abdomen positioning images as an output set.

The scanning range determining module 303 is configured to calculate an abdomen scanning range and a scanning center position based on the current liver outline information, and to apply the scanning range and scanning center position in a current scanning protocol so that an MRI device can use the scanning protocol to perform a magnetic resonance scan of an abdomen.

In one embodiment, the system may further comprise the following, as shown by the dotted-line part in FIG. 3: a navigation bar position determining module 304 configured to calculate a liver center-of-mass position or a liver outline incircle center position based on the current liver outline information, calculate a navigation bar position required for respiratory navigation according to the liver center-of-mass position or the liver outline incircle center position, and to apply the navigation bar position in the current scanning protocol.

In one embodiment, the abdomen positioning image comprises a sagittal image, a transverse image and a coronal image; and the liver outline information comprises: a dimension in the head-feet direction, a dimension in the anterior-posterior direction, and a dimension in the left-right direction, taking the patient as the positioning center.

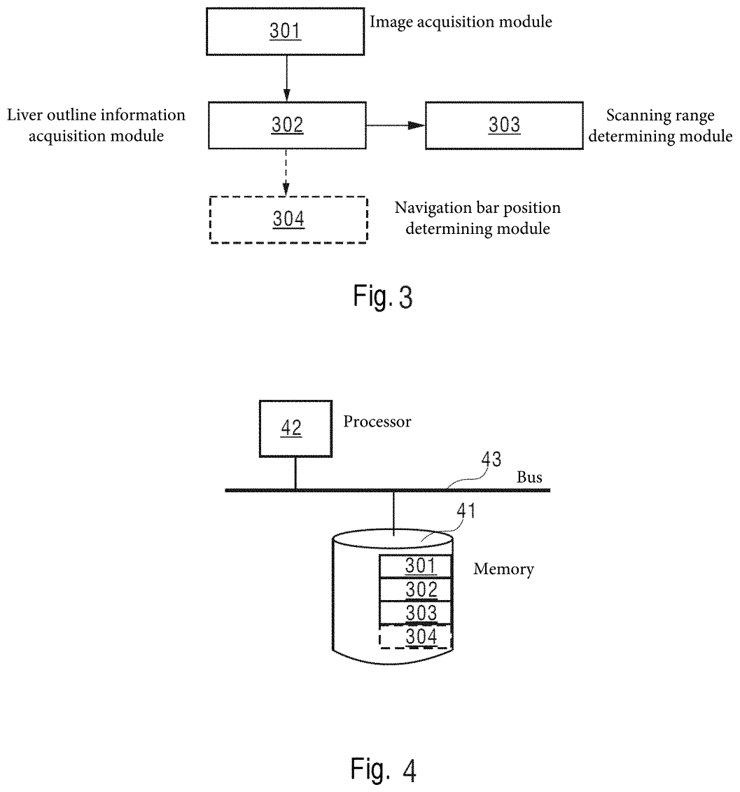

FIG. 4 illustrates a demonstrative structural diagram of another abdomen scanning system in an embodiment of the present disclosure. As shown in FIG. 4, the apparatus may comprise: at least one memory 41 and at least one processor 42. In addition, some other components may also be included, such as a communication port, etc. These components communicate via a bus 43.

The at least one memory 41 is configured to store a computer program. In one embodiment, the computer program may be understood to comprise the various modules of the abdomen scanning system as shown in FIG. 3. Furthermore, the at least one memory 41 may also store an operating system, etc. Operating systems include but are not limited to: an Android operating system, a Symbian operating system, a Windows operating system, and a Linux operating system, etc.

The at least one processor 42 is configured to call (i.e. execute) the computer program stored in the at least one memory 41 to perform the abdomen scanning method described in an embodiment of the present disclosure. The processor 42 may be implemented as any suitable type of processor and/or processing circuitry such as, for example, a CPU, a processing unit/module, an ASIC, a logic module or a programmable gate array, etc. The processor 42 may receive and/or send data via the communication port or any other suitable means.

It is explained that not all of the steps and modules in the flows and structural diagrams above are necessary; certain steps or modules may be omitted according to actual requirements. The order in which steps are executed is not fixed, but may be adjusted as required. The partitioning of the modules is merely functional partitioning, employed for the purpose of facilitating description; during actual implementation, one module may be realized by multiple modules, and the functions of multiple modules may be realized by the same module; these modules may be located in the same device, or in different devices.

It can be understood that hardware modules in the embodiments above may be realized mechanically or electronically. For example, one hardware module may comprise a specially designed permanent circuit or logic device (such as a dedicated processor, such as an FPGA or ASIC) for completing a specific operation. The hardware module may also comprise a programmable logic device or circuit that is temporarily configured by software (e.g. comprising a general processor or another programmable processor) for executing a specific operation. The choice of whether to specifically use a mechanical method, or a dedicated permanent circuit, or a temporarily configured circuit (e.g. configured by software) to realize the hardware module can be decided according to considerations of cost and time.

In addition, in an embodiment of the present disclosure, a computer readable storage medium (e.g. memory 41) is further provided, having stored thereon a computer program that can be executed by a processor and realize the abdomen scanning method described in embodiments of the present disclosure. Specifically, a system or apparatus equipped with a storage medium may be provided; software program code realizing the function of any one of the embodiments above is stored on the storage medium, and a computer (or CPU or MPU) of the system or apparatus is caused to read and execute the program code stored in the storage medium. Furthermore, it is also possible to cause an operating system, etc. operating on a computer to complete a portion of, or all, actual operations by means of an instruction based on program code. It is also possible for program code read out from the storage medium to be written into a memory installed in an expansion board inserted in the computer, or written into a memory installed in an expansion unit connected to the computer, and thereafter instructions based on the program code cause a CPU etc. installed on the expansion board or expansion unit to execute a portion of and all actual operations, so as to realize the function of any one of the embodiments above. Embodiments of storage media used for providing program code include floppy disks, hard disks, magneto-optical disks, optical disks (such as CD-ROM, CD-R, CD-RW, DVD-ROM, DVD-RAM, DVD-RW, DVD+RW), magnetic tapes, non-volatile memory cards and ROM. Optionally, program code may be downloaded from a server computer via a communication network.

It can be seen from the solution above that in embodiments of the present disclosure, a liver outline acquisition model is obtained by training in advance based on an artificial intelligence network using a first quantity of historical abdomen positioning images as an input set, and using a first quantity of historical liver outline information respectively corresponding to the first quantity of historical abdomen positioning images as an output set, and liver region outline information is acquired based on the liver outline acquisition model; and a scanning range is determined based on the acquired liver region outline information. As the liver outline information acquired by such a method is relatively accurate, it can increase the accuracy of automatic identification of the abdomen scanning range.

In addition, by determining a liver center-of-mass position or a liver outline incircle center position based on the acquired liver region outline information, and determining a navigation bar position based on the liver center-of-mass position or liver outline incircle center position, it is possible to ensure that the navigation bar position is within the outline of the liver region, thus increasing the accuracy of navigation bar positioning.

The embodiments above are merely preferred embodiments of the present disclosure, which are not intended to limit it. Any amendments, equivalent substitutions or improvements, etc. made within the spirit and principles of the present disclosure shall be included in the scope of protection thereof.

The various components described herein may be referred to as “modules.” Such components may be implemented via any suitable combination of parts, components, hardware, and/or software components as applicable and/or known to achieve the intended functionality of the respective modules. Again, this may include mechanical and/or electrical components, FPGAs, processors, processing circuitry, or other suitable hardware components configured to execute instructions or computer programs that are stored on a suitable computer readable medium. Regardless of the particular implementation, such modules when applicable and relevant may alternatively be referred to herein as “circuitry,” “processors,” or “processing circuitry.”

What is claimed is:

1. An abdomen scanning method, comprising:
acquiring a current abdomen positioning image;
inputting the current abdomen positioning image into a pre-trained liver outline acquisition model;
obtaining current liver outline information output by the liver outline acquisition model, wherein the liver outline acquisition model is trained using (i) a first quantity of historical abdomen positioning images as an input set, and (ii) a first quantity of historical liver outline information respectively corresponding to the first quantity of historical abdomen positioning images as an output set;
based on the current liver outline information, calculating an abdomen scanning range and a scanning center position;
applying the scanning range and scanning center position as part of a magnetic resonance (MR) scanning protocol; and
using the MR scanning protocol to perform a MR scan of an abdomen of a patient.

2. The abdomen scanning method as claimed in claim 1, further comprising:
before using the scanning protocol to perform the MR scan of the abdomen of the patient:
calculating a liver center-of-mass position or a liver outline incricle center position based on the current liver outline information;
calculating a navigation bar position for respiratory navigation according to the liver center-of-mass position or the liver outline incricle center position; and
applying the navigation bar position in the MR scanning protocol.

3. The abdomen scanning method as claimed in claim 1, wherein the abdomen positioning image comprises a sagittal image, a transverse image, and a coronal image, and
wherein the liver outline information comprises, with respect to the patient as a positioning center, (i) a dimension in a head-to-feet direction of the patient, (ii) a dimension in an anterior-posterior direction, and (iii) a dimension in a left-right direction.

4. An abdomen scanning system, comprising:
image acquisition circuitry configured to acquire a current abdomen positioning image;
liver outline information acquisition circuitry configured to input the current abdomen positioning image into a pre-trained liver outline acquisition model, and to obtain current liver outline information output by the liver outline acquisition model,
wherein the liver outline acquisition model is trained using (i) a first quantity of historical abdomen positioning images as an input set, and (ii) a first quantity of historical liver outline information respectively corresponding to the first quantity of historical abdomen positioning images as an output set;
scanning range determining circuitry configured to calculate an abdomen scanning range and a scanning center position based on the current liver outline information, and to apply the scanning range and scanning center position in a magnetic resonance (MR) scanning protocol such that a magnetic resonance imaging (MRI) device executes the MR scanning protocol to perform a MR scan of an abdomen of a patient.

5. The abdomen scanning system as claimed in claim 4, further comprising:
navigation bar position determining circuitry configured to calculate a liver center-of-mass position or a liver outline incricle center position based on the current liver outline information to calculate a navigation bar position required for respiratory navigation according to the liver center-of-mass position or the liver outline incricle center position, and to apply the navigation bar position in the MR scanning protocol.

6. The abdomen scanning system as claimed in claim 4, wherein the abdomen positioning image comprises a sagittal image, a transverse image, and a coronal image, and
wherein the liver outline information comprises, with respect to the patient as a positioning center, (i) a dimension in the head-to-feet direction, (ii) a dimension in the anterior-posterior direction, and (iii) a dimension in the left-right direction.

7. An abdomen scanning system, comprising:
a memory configured to store a computer program; and
a processor configured to execute the computer program stored in the memory to cause the abdomen scanning system to:
acquire a current abdomen positioning image;
input the current abdomen positioning image into a pre-trained liver outline acquisition model;
obtain current liver outline information output by the liver outline acquisition model, wherein the liver outline acquisition model is trained using (i) a first quantity of historical abdomen positioning images as an input set, and (ii) a first quantity of historical liver outline information respectively corresponding to the first quantity of historical abdomen positioning images as an output set;
based on the current liver outline information, calculate an abdomen scanning range and a scanning center position;
apply the scanning range and scanning center position as part of a magnetic resonance (MR) scanning protocol; and
using the MR scanning protocol to perform a MR scan of an abdomen of a patient.

8. The abdomen scanning system as claimed in claim 7, wherein the computer program stored on the memory is executed by the processor to cause the abdomen scanning system to, before using the scanning protocol to perform the magnetic resonance scan of the abdomen:
calculate a liver center-of-mass position based on the current liver outline information;
calculate a navigation bar position for respiratory navigation according to the liver center-of-mass position; and
apply the navigation bar position in the MR scanning protocol.

9. The abdomen scanning system as claimed in claim 7, wherein the abdomen positioning image comprises a sagittal image, a transverse image, and a coronal image, and
wherein the liver outline information comprises, with respect to the patient as a positioning center, (i) a dimension in a head-to-feet direction of the patient, (ii) a dimension in an anterior-posterior direction, and (iii) a dimension in a left-right direction.
